(19) United States
(12) Patent Application Publication
(10) Pub. No.: US 2026/0199586 A1
(43) Pub. Date: Jul. 16, 2026

(54) NEGATIVE PRESSURE WOUND THERAPY DEVICES AND METHODS UTILIZING A TURBINE

(71) Applicant: T.J.Smith and Nephew,Limited, Hull (GB)

(72) Inventors: Adam John Fisher, Hull (GB); Benjamin James Gardner, Hull (GB); Allan Kenneth Frazer Grugeon Hunt, Beverley (GB); Andrea Maggiore, Hull (GB); Kirsty Margaret Strachan, Hull (GB)

(21) Appl. No.: 19/135,539

(22) PCT Filed: Jan. 30, 2024
(86) PCT No.: PCT/EP2024/052137

§ 371 Date: Jun. 4, 2025

(30) Foreign Application Priority Data
Jan. 31, 2023 (GB) 2301329.5

(51) Int. Cl. A61M 1/00

(52) U.S. Cl. CPC A61M 1/982; A61M 1/96; A61M 2205/3317; A61M 2205/3334; A61M 2205/3344; A61M 2205/3365

(57) ABSTRACT

A negative pressure wound therapy system can include a source of negative pressure configured to be fluidically connected by a fluid flow path to a wound covered by a wound dressing and further configured to provide negative pressure to the wound and a turbine configured to be positioned in the fluid flow path and further configured to monitor a rate of a flow of fluid in the fluid flow path responsive to a rotation of the turbine caused by the flow of fluid.

### TECHNICAL FIELD

Embodiments described herein relate to apparatuses, systems, and methods for the treatment of wounds, for example using dressings in combination with negative pressure wound therapy.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY

A negative pressure wound therapy system can include a source of negative pressure configured to be fluidically connected by a fluid flow path to a wound covered by a wound dressing and further configured to provide negative pressure to the wound. The system can include a canister configured to be positioned in the fluid flow path and further configured to store fluid aspirated from the wound as a result of negative pressure being provided to the wound by the source of negative pressure. The system can include a turbine configured to be positioned in the fluid flow path and further configured to monitor a rate of a flow of fluid in the fluid flow path responsive to a rotation of the turbine caused by the flow of fluid.

The negative pressure wound therapy system of any of the preceding paragraphs and/or any of the systems, apparatuses, or devices disclosed herein can include one or more of the following features. The turbine can include a wheel with a plurality of fins. The system can include at least two magnets attached to the plurality of fins. The system can include a Hall effect sensor configured to interact with the at least two magnets to monitor a rotational speed of the turbine. The turbine can be configured to monitor the rate of flow of fluid responsive to detection of the rotational speed. The at least two magnets can be spaced equally on the wheel of the turbine.

The negative pressure wound therapy system of any of the preceding paragraphs and/or any of the systems, apparatuses, or devices disclosed herein can include one or more of the following features. The turbine can be positioned in the canister, a lumen configured to connect the source of negative pressure to the wound, or a wound dressing port configured to connect the source of negative pressure to the wound. Rotation of the turbine can causes an energy to be produced. The amount of energy can be dependent on a viscosity of fluid flowing in the fluid flow path. The system can include an electronic processing circuitry configured to determine the viscosity of fluid based on the amount of energy. The system can include a sensor. The energy can be used to power the sensor. The system can include an electronic processing circuitry configured to provide an indication of the rate of flow of fluid.

The negative pressure wound therapy system of any of the preceding paragraphs and/or any of the systems, apparatuses, or devices disclosed herein can include one or more of the following features. The system can include an electronic processing circuitry configured to determine a pressure at the wound based on the rate of flow of fluid and provide an indication of the pressure at the wound. The electronic processing circuitry can be configured to determine the pressure at the wound based on the rate of flow of fluid, a viscosity of fluid flowing in the fluid flow path, and a level of negative pressure being provided by the source of negative pressure to the wound or being measured at a location in the fluid flow path. The rotation of the turbine can cause an energy to be produced, an amount of the energy being dependent on the viscosity of fluid flowing in the fluid flow path. The electronic processing circuitry can be configured to determine the viscosity of fluid based on the amount of energy. The electronic processing circuitry can be configured to determine the pressure at the wound based on a length, radius, and cross-sectional area of a lumen configured to connect the source of negative pressure to the wound.

The negative pressure wound therapy system of any of the preceding paragraphs and/or any of the systems, apparatuses, or devices disclosed herein can include one or more of the following features. The turbine can be positioned in an inlet of the canister. The system can include an electronic processing circuitry configured to determine that the canister is full responsive to detecting a lack of rotation of the turbine and provide an indication that the canister is full. An axis of rotation of the turbine can be parallel or perpendicular to a level of fluid in the canister.

The negative pressure wound therapy system of any of the preceding paragraphs and/or any of the systems, apparatuses, or devices disclosed herein can include one or more of the following features. The system can include an electronic processing circuitry configured to determine a level of fluid in the canister based on the rate of flow of fluid and provide an indication of the level of fluid in the canister. The electronic processing circuitry can be configured to determine the level of fluid in the canister based on the rate of flow of fluid and a volume of the canister. The electronic processing circuitry can be configured to determine that the canister is full responsive to determining that the level of fluid in the canister reaches the volume of the canister. The electronic processing circuitry can be configured to determine the level of fluid in the canister based on monitoring the rate of flow of fluid over a duration of time.

A negative pressure wound therapy system can include a source of negative pressure configured to be fluidically connected by a fluid flow path to a wound covered by a wound dressing and further configured to provide negative pressure to the wound. The system can include a turbine configured to be positioned in the fluid flow path and further configured to monitor a rate of a flow of fluid in the fluid flow path responsive to a rotation of the turbine caused by the flow of fluid.

The negative pressure wound therapy system of any of the preceding paragraphs and/or any of the systems, apparatuses, or devices disclosed herein can include one or more of the following features. Rotation can be monitored in in response to at least one of: change in a magnetic field caused by movement of a magnet attached to the turbine, change in an electric field caused by movement of a conductive component positioned on the turbine, change in a resistance caused by movement of the turbine, movement of a wireless tag attached to the turbine, movement of a protrusion of the turbine, change of color, or movement of an infrared emitter or sensor supported by on the turbine.

The negative pressure wound therapy system of any of the preceding paragraphs and/or any of the systems, apparatuses, or devices disclosed herein can include one or more of the following features. The turbine can include a wheel with a plurality of fins. The system can include at least two magnets attached to the plurality of fins. The system can include a Hall effect sensor configured to interact with the at least two magnets to monitor a rotational speed of the turbine. The turbine can be configured to monitor the rate of flow of fluid responsive to detection of the rotational speed.

The negative pressure wound therapy system of any of the preceding paragraphs and/or any of the systems, apparatuses, or devices disclosed herein can include one or more of the following features. Rotation of the turbine can cause an energy to be produced. An amount of the energy can be dependent on a viscosity of fluid flowing in the fluid flow path. The system can include an electronic processing circuitry configured to determine the viscosity of fluid based on the amount of energy.

The negative pressure wound therapy system of any of the preceding paragraphs and/or any of the systems, apparatuses, or devices disclosed herein can include one or more of the following features. The system can include an electronic processing circuitry configured to determine a pressure at the wound based on the rate of flow of fluid and provide an indication of the pressure at the wound.

The negative pressure wound therapy system of any of the preceding paragraphs and/or any of the systems, apparatuses, or devices disclosed herein can include one or more of the following features. The system can include a canister configured to be positioned in the fluid flow path and further configured to store fluid aspirated from the wound as a result of negative pressure being provided to the wound by the source of negative pressure. The system can include an electronic processing circuitry configured to at least one of: determine that the canister is full responsive to detecting a lack of rotation of the turbine or determine a level of fluid in the canister based on the rate of flow of fluid.

Disclosed herein are methods of operating a negative pressure wound therapy device of any of the preceding paragraphs and/or any of the devices, apparatuses, or systems disclosed herein.

Disclosed herein are kits that include the negative pressure wound therapy device of any of the preceding paragraphs and/or any of the devices, apparatuses, or systems disclosed herein and one or more wound dressings.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1A illustrates a negative pressure wound therapy system.

FIG. 1B illustrates another negative pressure wound therapy system.

FIG. 2A is an isometric view of a negative pressure wound therapy device and canister, showing the canister detached from the pump assembly of the device.

FIG. 2B is a back view of the negative pressure wound therapy device shown in FIG. 2A.

FIG. 2C illustrates a top surface of the negative pressure wound therapy device shown in FIG. 2A, showing a user interface.

FIG. 2D illustrates a side view of the negative pressure wound therapy device shown in FIG. 2A.

FIG. 3 illustrates a schematic of a control system of a negative pressure wound therapy device.

FIG. 4 illustrates another negative pressure wound therapy system.

FIGS. 5A-5C illustrate various views of a negative pressure wound therapy device with a turbine assembly.

FIG. 6 illustrates a positioning of a turbine assembly in the canister.

FIG. 7A illustrates a turbine assembly with a magnetic sensing system.

FIG. 7B illustrates a turbine assembly with a mechanical triggering system.

FIG. 7C illustrates a turbine assembly with a photo sensing system.

FIG. 7D illustrates a turbine assembly with a proximity sensing system.

FIG. 7E illustrates a turbine assembly with an electrically conductive sensing system.

FIG. 7F illustrates a turbine assembly with a communication tag sensing system.

FIG. 8 illustrates a turbine assembly electrically coupled to a negative pressure wound therapy device.

### DETAILED DESCRIPTION

Embodiments disclosed herein relate to systems and methods of treating and/or monitoring a wound. Some embodiments of the negative pressure wound therapy devices disclosed herein can include a negative pressure source configured to be connected and/or fluidically coupled, via a fluid flow path, to a wound covered by a wound dressing and provide negative pressure to a wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, either as a result of surgery, trauma, sternotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760-X) mmHg. In addition, negative pressure that is “less” or “smaller” than X mmHg corresponds to pressure that is closer to atmospheric pressure (for example, −40 mmHg is less than −60 mmHg). Negative pressure that is “more” or “greater” than −X mmHg corresponds to pressure that is further from atmospheric pressure (for example, −80 mmHg is more than −60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Embodiments disclosed herein provide systems, devices, or methods of efficiently adjusting TNP prescriptions and delivering effective TNP therapy.

### Wound Therapy System

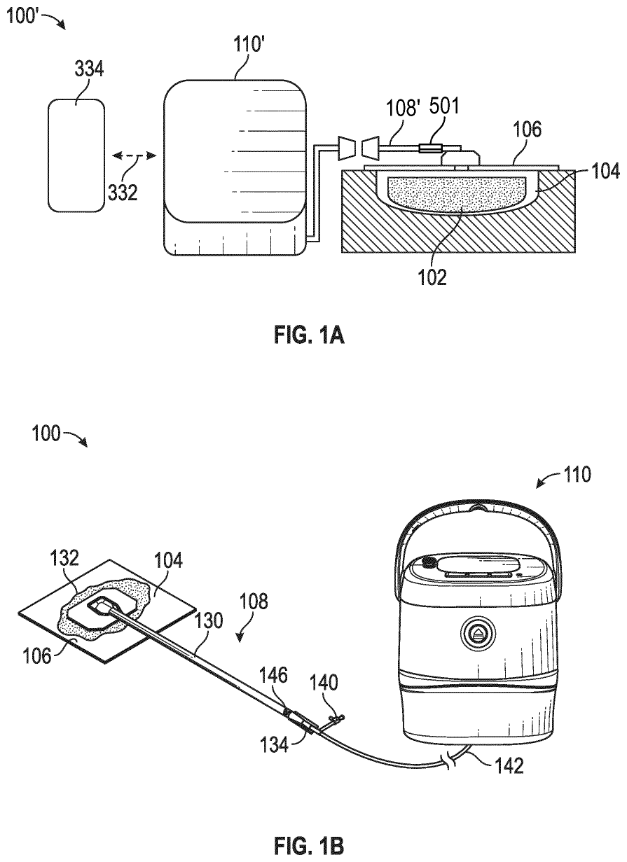

FIG. 1A schematically illustrates a negative pressure wound treatment system 100′ (sometimes referred to as a reduced or negative pressure wound therapy system, a TNP system, or a wound treatment system). In any implementations disclosed herein, though not required, the negative pressure wound treatment system 100′ can include a wound filler 102 placed on or inside a wound 104 (which may be a cavity). The wound 104 can be sealed by a wound cover 106, which can be a drape, such that the wound cover 106 can be in fluidic communication with the wound 104. The wound filler 102 in combination with the wound cover 106 can be referred to as a wound dressing. A tube or conduit 108′ (also referred to herein as a flexible suction adapter or a fluidic connector) can be used to connect the wound cover 106 with a wound therapy device 110′ (sometimes as a whole or partially referred to as a “pump assembly”) configured to supply reduced or negative pressure. The conduit 108′ can be a single or multi lumen tube. A connector can be used to removably and selectively couple a conduit or tube of the device 110′ with the conduit 108′.

In any of the systems disclosed herein, a wound therapy device can be canisterless, wherein, for example and without limitation, wound exudate is collected in the wound dressing or is transferred via a conduit for collection at another location. However, any of the wound therapy devices disclosed herein can include or support a canister.

Additionally, with any of the wound therapy systems disclosed herein, any of the wound therapy devices can be mounted to or supported by the wound dressing or adjacent to the wound dressing. The wound filler 102 can be any suitable type, such as hydrophilic or hydrophobic foam, gauze, inflatable bag, and so on. The wound filler 102 can be conformable to the wound 104 such that the wound filler 102 substantially fills the cavity of the wound 104. The wound cover 106 can provide a substantially fluid impermeable seal over the wound 104. The wound cover 106 can have a top side and a bottom side. The bottom side can adhesively (or in any other suitable manner) seal with the wound 104, for example by sealing with the skin around the wound 104. The conduit 108 or any other conduit disclosed herein can be formed from polyurethane, PVC, nylon, polyethylene, silicone, or any other suitable material.

The wound cover 106 can have a port (not shown) configured to receive an end of the conduit 108. In some cases, the conduit 108 can otherwise pass through or under the wound cover 106 to supply reduced pressure to the wound 104 so as to maintain a desired level of reduced pressure in the wound 104. The conduit 108 can be any suitable article configured to provide at least a substantially sealed fluid flow pathway or path between the wound therapy device 110′ and the wound cover 106, so as to supply the reduced pressure provided by the wound therapy device 110′ to wound 104. As described herein, a turbine assembly 501 can be positioned in the conduit 108. The turbine assembly 501 can be utilized to determine one or more of a flow rate of fluid in the fluid flow path, viscosity (or type) of fluid, pressure drop, fluid level, canister full, leak, blockage, or the like.

The wound cover 106 and the wound filler 102 can be provided as a single article or an integrated single unit. In some cases, no wound filler is provided and the wound cover by itself may be considered the wound dressing. The wound dressing can then be connected, via the conduit 108, to a source of negative pressure of the wound therapy device 110′. In some cases, though not required, the wound therapy device 110′ can be miniaturized and portable, although larger conventional negative pressure sources (or pumps) can also be used.

The wound cover 106 can be located over a wound site to be treated. The wound cover 106 can form a substantially sealed cavity or enclosure over the wound. The wound cover 106 can have a film having a high water vapour permeability to enable the evaporation of surplus fluid, and can have a superabsorbing material contained therein to safely absorb wound exudate. In some cases, the components of the TNP systems described herein can be particularly suited for incisional wounds that exude a small amount of wound exudate.

The wound therapy device 110′ can operate with or without the use of an exudate canister. In some cases, as is illustrated, the wound therapy device 110′ can include an exudate canister. In some cases, configuring the wound therapy device 110′ and conduit 108′ so that the conduit 108′ can be quickly and easily removed from the wound therapy device 110′ can facilitate or improve the process of wound dressing or pump changes, if necessary. Any of the pump assemblies disclosed herein can have any suitable connection between the conduit 108′ and the pump.

The wound therapy device 110′ can deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also, in some cases a pressure range of below −75 mmHg can be used. Alternatively, a pressure range of over approximately −100 mmHg, or even −150 mmHg, can be supplied by the wound therapy device 110′.

As will be described in greater detail below, the negative pressure wound treatment system 100′ can be configured to provide a connection 332 to a separate or remote computing device 334. The connection 332 can be wired or wireless (such as, Bluetooth, Bluetooth low energy (BLE), Near-Field Communication (NFC), WiFi, or cellular). The remote computing device 334 can be a smartphone, a tablet, a laptop or another standalone computer, a server (such as, a cloud server), another pump device, or the like.

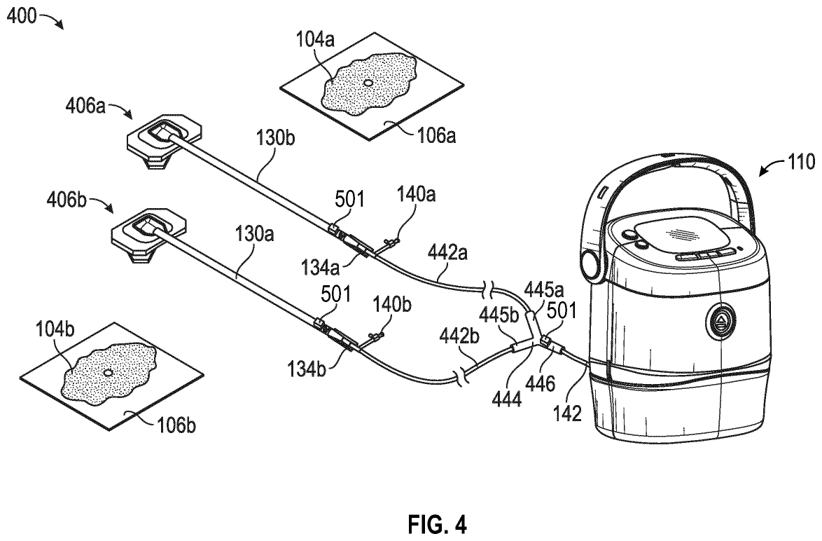

FIG. 1B illustrates another negative pressure wound treatment system 100. The negative pressure wound treatment system 100 can have any of the components, features, or other details of any of the other negative pressure wound treatment system disclosed herein, including without limitation the negative pressure wound treatment system 100′ illustrated in FIG. 1A or the negative pressure wound treatment system 400 illustrated in FIG. 4, in combination with or in place of any of the components, features, or other details of the negative pressure wound treatment system 100 shown in FIG. 1B and/or described herein. The negative pressure wound treatment system 100 can have a wound cover 106 over a wound 104 that can seal the wound 104. A conduit 108, such as a single or multi lumen tube can be used to connect the wound cover 106 with a wound therapy device 110 (sometimes as a whole or partially referred to as a “pump assembly”) configured to supply reduced or negative pressure. The wound cover 106 can be in fluidic communication with the wound 104.

With reference to FIG. 1B, the conduit 108 can have a bridge portion 130 that can have a proximal end portion and a distal end portion the distal end portion being closer to the wound 104 than the proximal end portion, and an applicator 132 at the distal end of the bridge portion 130 forming the flexible suction adapter (or conduit) 108. A connector 134 can be disposed at the proximal end of the bridge portion 130, so as to connect to at least one of the channels that can extend along a length of the bridge portion 130 of the conduit 108 shown in FIG. 1B. A cap 140 can be coupled with a portion of the conduit 108 and can, in some cases, as illustrated, be attached to the connector 134. The cap 140 can be useful in preventing fluids from leaking out of the proximal end of the bridge portion 130. The conduit 108 can be a Soft Port manufactured by Smith & Nephew. As mentioned, the negative pressure wound treatment system 100 can include a source of negative pressure, such as the device 110, capable of supplying negative pressure to the wound 104 through the conduit 108. Though not required, the device 110 can also include a canister or other container for the storage of wound exudates and other fluids that can be removed from the wound.

The device 110 can be connected to the connector 134 via a conduit or tube 142. In use, the applicator 132 can be placed over an aperture formed in a cover 106 that is placed over a suitably-prepared wound or wound 104. Subsequently, with the wound therapy device 110 connected via the tube 142 to the connector 134, the wound therapy device 110 can be activated to supply negative pressure to the wound. Application of negative pressure can be applied until a desired level of healing of the wound is achieved.

The bridge portion 130 can comprise an upper channel material or layer positioned between an upper layer and an intermediate layer, with a lower channel material or layer positioned between the intermediate layer and a bottom layer. The upper, intermediate, and lower layers can have elongate portions extending between proximal and distal ends and can include a material that is fluid-impermeable, for example polymers such as polyurethane. It will of course be appreciated that the upper, intermediate, and lower layers can each be constructed from different materials, including semi-permeable materials. In some cases, one or more of the upper, intermediate, and lower layers can be at least partially transparent. In some instances, the upper and lower layers can be curved, rounded or outwardly convex over a majority of their lengths.

The upper and lower channel layers can be elongate layers extending from the proximal end to the distal end of the bridge 130 and can each preferably comprise a porous material, including for example open-celled foams such as polyethylene or polyurethane. In some cases, one or more of the upper and lower channel layers can be comprised of a fabric, for example a knitted or woven spacer fabric (such as a knitted polyester 3D fabric, Baltex 7970®, or Gehring 879®) or a nonwoven material, or terry-woven or loop-pile materials. The fibers may not necessarily be woven, and can include felted and flocked (including materials such as Flotex®) fibrous materials. The materials selected are preferably suited to channeling wound exudate away from the wound and for transmitting negative pressure or vented air to the wound site, and can also confer a degree of kinking or occlusion resistance to the channel layers. In one example, the upper channel layer can include an open-celled foam such as polyurethane, and the lower channel layer can include a fabric. In another example, the upper channel layer is optional, and the system can instead be provided with an open upper channel. The upper channel layer can have a curved, rounded or upwardly convex upper surface and a substantially flat lower surface, and the lower channel layer can have a curved, rounded or downwardly convex lower surface and a substantially flat upper surface.

The fabric or material of any components of the bridge 130 can have a three-dimensional (3D) structure, where one or more types of fibers form a structure where the fibers extend in all three dimensions. Such a fabric can in some cases aid in wicking, transporting fluid or transmitting negative pressure. In some cases, the fabric or materials of the channels can include several layers of material stacked or layered over each other, which can in some cases be useful in preventing the channel from collapsing under the application of negative pressure. The materials used in some implementations of the conduit 108 can be conformable and pliable, which can, in some cases, help to avoid pressure ulcers and other complications which can result from a wound treatment system being pressed against the skin of a patient.

The distal ends of the upper, intermediate, and lower layers and the channel layers can be enlarged at their distal ends (to be placed over a wound site), and can form a “teardrop” or other enlarged shape. The distal ends of at least the upper, intermediate, and lower layers and the channel layers can also be provided with at least one through aperture. This aperture can be useful not only for the drainage of wound exudate and for applying negative pressure to the wound, but also during manufacturing of the device, as these apertures can be used to align these respective layers appropriately.

In some implementations, a controlled gas leak 146 (sometimes referred to as gas leak, air leak, or controlled air leak) can be disposed on the bridge portion 130, for example at the proximal end thereof. This air leak 146 can comprise an opening or channel extending through the upper layer of the bridge portion 130, such that the air leak 146 is in fluidic communication with the upper channel of the bridge portion 130. Upon the application of suction to the conduit 108, gas (such, as air) can enter through the gas leak 146 and move from the proximal end of the bridge portion 130 to the distal end of the bridge portion along the upper channel of the bridge portion 130. The gas can then be suctioned into the lower channel of the bridge portion 130 by passing through the apertures through the distal ends of the upper, intermediate, and lower layers.

The air leak 146 can include a filter. Preferably, the air leak 146 is located at the proximal end of the bridge portion 130 so as to minimize the likelihood of wound exudate or other fluids coming into contact and possibly occluding or interfering with the air leak 146 or the filter. In some instances, the filter can be a microporous membrane capable of excluding microorganisms and bacteria, and which may be able to filter out particles larger than 45 μm. Preferably, the filter can exclude particles larger than 1.0 μm, and more preferably, particles larger than 0.2 μm. Advantageously, some implementations can provide for a filter that is at least partially chemically-resistant, for example to water, common household liquids such as shampoos, and other surfactants. In some cases, reapplication of vacuum to the suction adapter or wiping of the exposed outer portion of the filter may be sufficient to clear any foreign substance occluding the filter. The filter can be composed of a suitably-resistant polymer such as acrylic, polyethersulfone, or polytetrafluoroethylene, and can be oleophobic or hydrophobic. In some cases, the gas leak 146 can supply a relatively constant gas flow that does not appreciably increase as additional negative pressure is applied to the conduit 108. In instances of the negative pressure wound treatment system 100 where the gas flow through the gas leak 146 increases as additional negative pressure is applied, preferably this increased gas flow will be minimized and not increase in proportion to the negative pressure applied thereto. Further description of such bridges, conduits, air leaks, and other components, features, and details that can be used with any implementations of the negative pressure wound treatment systems disclosed herein are found in U.S. Pat. No. 8,801,685, which is incorporated by reference in its entirety as if fully set forth herein.

Any of the wound therapy devices (such as, the device 110 or 110′) disclosed herein can provide continuous or intermittent negative pressure therapy. Continuous therapy can be delivered at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. Intermittent therapy can be delivered between low and high negative pressure set points (sometimes referred to as setpoint). Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, or below −180 mmHg. High set point can be set at above −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values.

In operation, the wound filler 102 can be inserted into the cavity of the wound 104, and wound cover 106 can be placed so as to seal the wound 104. The wound therapy device 110′ can provide negative pressure to the wound cover 106, which can be transmitted to the wound 104 via the wound filler 102. Fluid (such as, wound exudate) can be drawn through the conduit 108′ and stored in a canister. In some cases, fluid is absorbed by the wound filler 102 or one or more absorbent layers (not shown).

Wound dressings that can be utilized with the pump assembly and systems of the present application include Renasys-F, Renasys-G, Renasys AB, and Pico Dressings available from Smith & Nephew. Further description of such wound dressings and other components of a negative pressure wound therapy system that can be used with the pump assembly and systems of the present application are found in U.S. Patent Publication Nos. 2012/0116334, 2011/0213287, 2011/0282309, 2012/0136325, U.S. Pat. No. 9,084,845, and International App. No. PCT/EP2020/078376, each of which is incorporated by reference in its entirety as if fully set forth herein. In some cases, other suitable wound dressings can be utilized.

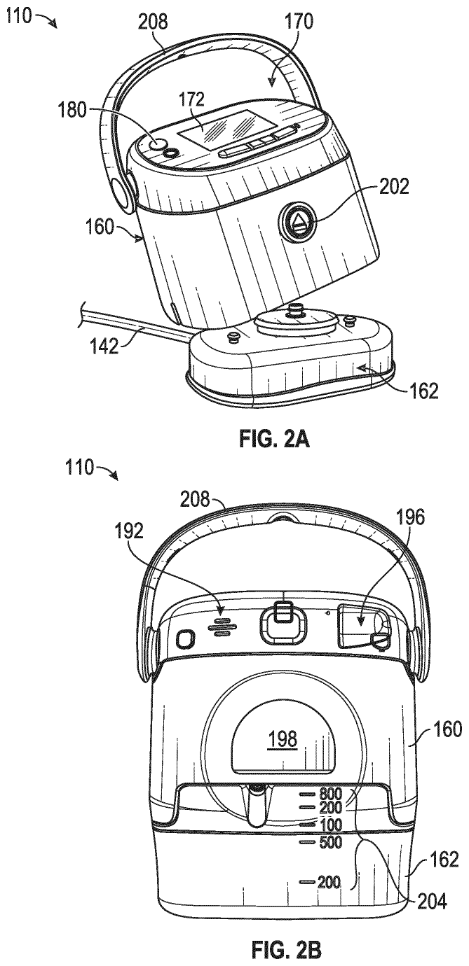

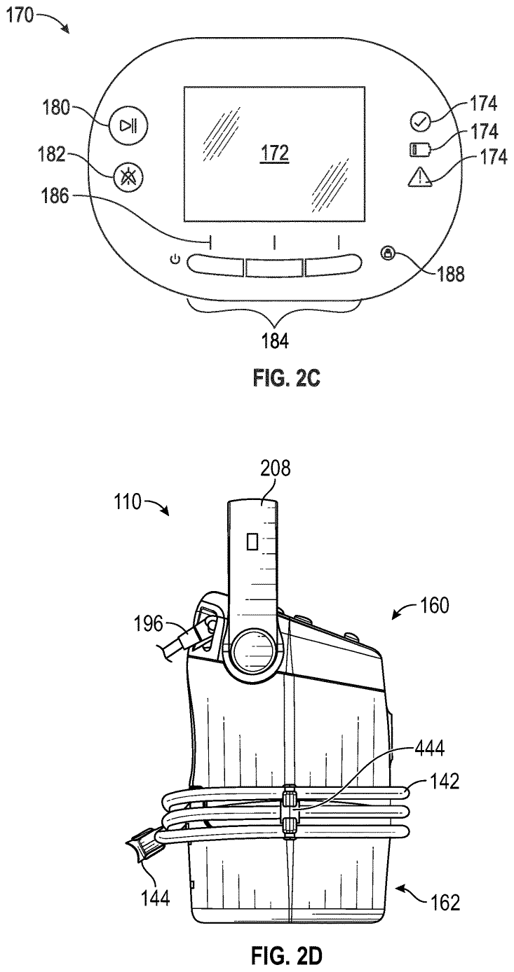

FIGS. 2A-2D show the negative pressure wound therapy device 110. As illustrated, a pump assembly 160 and canister 162 can be connected, thereby forming the wound therapy device 110. With reference to FIG. 2C, the pump assembly 160 can include an interface panel 170 having a display 172, one or more indicators 174, or one or more controls or buttons, including, for example and without limitation, a therapy start and pause button 180 or an alarm/alert mute button 182. The interface panel 170 can have one or more input controls or buttons 184 (three being shown) that can be used to control any functions of the pump assembly 160 or the interface panel 170. For example and without limitation, one or more of the buttons 184 can be used to turn the pump assembly 160 on or off, to start or pause therapy, to operate and monitor the operation of the pump assembly 160, to scroll through menus displayed on the display 172, or to control or perform other functions. In some cases, the command buttons 184 can be programmable, and can be made from a tactile, soft rubber.

Additionally, the interface panel 170 can have visual indicators 186 that can indicate which of the one or more buttons 184 is active. The interface panel 170 can also have a lock/unlock control or button 188 that can be configured to selectively lock or unlock the functionality of the various buttons (e.g., buttons 184) or the display 172. For example, therapy setting adjustment can be locked/unlocked via the lock/unlock control 188. When the lock/unlock button 188 is in the locked state, depressing one or more of the various other buttons or the display will not cause the pump assembly 160 to change any display functions or performance functions of the device. This way, the interface panel 170 will be protected from inadvertent bumping or touching of the various buttons or display. The interface panel 170 can be located on an upper portion of the pump assembly 160, for example and without limitation on an upward facing surface of the pump assembly 160.

The display 172, which can be a screen such as an LCD screen, can be mounted in a middle portion of the interface panel 170. The display 172 can be a touch screen display. The display 172 can support playback of audiovisual (AV) content, such as instructional videos, and render a number of screens or graphical user interfaces (GUIs) for configuring, controlling, and monitoring the operation of the pump assembly 160.

The one or more indicators 174 can be lights (such as, LEDs) and can be configured to provide a visual indication of alarm conditions and or a status of the pump. For example and without limitation, the one or more indicators 174 can be configured to provide a visual indication of a status of the pump assembly 160 or other components of the negative pressure wound treatment system 100, including without limitation the conduit 108 or the wound cover 106 (such as, to provide an indication of normal operation, low battery, a leak, canister full, blockage, overpressure, or the like). Any one or more suitable indicators can be additionally or alternatively used, such as visual, audio, tactile indicator, and so on.

FIG. 2B shows a back or rear view of the wound therapy device 110 shown in the FIG. 2A. As shown, the pump assembly 160 can include a speaker 192 for producing sound. For example and without limitation, the speaker 192 can generate an acoustic alarm in response to deviations in therapy delivery, non-compliance with therapy delivery, or any other similar or suitable conditions or combinations thereof. The speaker 192 can provide audio to accompany one or more instructional videos that can be displayed on the display 172.

The pump assembly 160 can be configured to provide easy access (such as, an access door on the casing of the pump assembly) to one or more filters of the pump assembly 160, such as antibacterial filters. This can enable a user (such as, a healthcare provider or patient) to more easily access, inspect or replace such filters. The pump assembly 160 can also include a power jack 196 for providing power to the pump assembly 160 or for charging and recharging an internal power source (such as, a battery). Some implementations of the pump assembly 160 can include a disposable or renewable power source, such as one or more batteries, so that no power jack is needed. The pump assembly 160 can have a recess 198 formed therein to facilitate gripping of the pump assembly 160.

The canister 162 can hold fluid aspirated from the wound 104. For example, the canister 162 can have an 800 mL (or approximately 800 mL) capacity, or from a 300 mL or less capacity to a 1000 mL or more capacity, or any capacity level in this range. The canister 162 can include a tubing for connecting to the conduit 108 in order to form a fluid flow path. The canister 162 can be replaced with another canister, such as when the canister 162 has been filled with fluid. With reference to FIG. 2A, the wound therapy device 110 can include a canister inlet tube 142 (also referred to herein as a dressing port connector) in fluid communication with the canister 162. For example and without limitation, the canister inlet tube 142 can be used to connect with the conduit 108.

The canister 162 can be selectively coupleable and removable from the pump assembly 160. With reference to FIG. 2A, in some cases, a canister release button 202 can be configured to selectively release the canister 162 from the pump assembly 160. With reference to FIG. 2B, the canister 162 can have one or more fill lines or graduations 204 to indicate to the user and amount of fluid or exudate stored within the canister 162.

The wound therapy device 110 can have a handle 208 that can be used to lift or carry the wound therapy device 110. The handle 208 can be coupled with the pump assembly 160 and can be rotatable relative to the wound therapy device 110 so that the handle can be rotated upward for lifting or carrying the wound therapy device 110 or the pump assembly 160, or rotated into a lower profile in a more compact position when the handle is not being used. In some cases, the handle 208 can be coupled with the pump assembly 160 in a fixed position. The handle 208 can be coupled with an upper portion of the pump assembly 160 or can be removable from the wound therapy device 110.

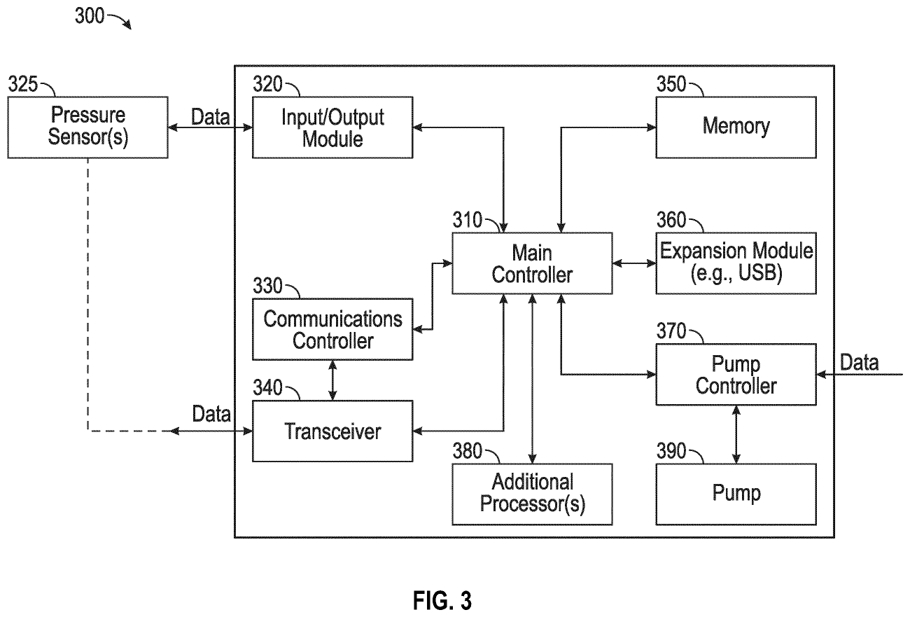

FIG. 3 illustrates a schematic of a control system 300 that can be employed in any of the wound therapy devices described herein, such as in the wound therapy device 110. Electrical components can operate to accept user input, provide output to the user, operate the pressure source, provide connectivity, and so on. A first processor (such as, a main controller 310) can be responsible for user activity, and a second processor (such as, a pump controller 370) can be responsible for controlling another device, such as a pump 390.

An input/output (I/O) module 320 can be used to control an input and/or output to another component or device, such as the pump 390, one or more sensors (for example, one or more pressure sensors 325 configured to monitor pressure in one or more locations of the fluid flow path), or the like. For example, the I/O module can receive data from one or more sensors through one or more ports, such as serial (for example, I2C), parallel, hybrid ports, and the like. Any of the pressure sensors can be part of the wound therapy device or the canister. In some cases, any of the pressure sensors 325 can be remote to the wound therapy device, such as positioned at or near the wound (for example, in the dressing or the conduit connecting the dressing to the wound therapy device). In such implementations, any of the remote pressure sensors can communicate with the I/O module over a wired connection or with one or more transceivers 340 over a wireless connection.

The main controller 310 can receive data from and provide data to one or more expansion modules 360, such as one or more USB ports, SD ports, Compact Disc (CD) drives, DVD drives, FireWire ports, Thunderbolt ports, PCI Express ports, and the like. The main controller 310, along with other controllers or processors, can store data in memory 350 (such as one or more memory modules), which can be internal or external to the main controller 310. Any suitable type of memory can be used, including volatile or non-volatile memory, such as RAM, ROM, magnetic memory, solid-state memory, Magnetoresistive random-access memory (MRAM), and the like.

The main controller 310 can be a general purpose controller, such as a low-power processor or an application specific processor. The main controller 310 can be configured as a “central” processor in the electronic architecture of the control system 300, and the main controller 310 can coordinate the activity of other processors, such as the pump controller 370, one or more communications controllers 330, and one or more additional processors 380. The main controller 310 can run a suitable operating system, such as a Linux, Windows CE, VxWorks, etc.

The pump controller 370 can control the operation of a pump 390, which can generate negative or reduced pressure. The pump 390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The pump controller 370 can measure pressure in a fluid flow path, using data received from one or more pressure sensors 325, calculate the rate of fluid flow, and control the pump. The pump controller 370 can control the pump actuator (such as, a motor) so that a desired level of negative pressure is achieved in the wound 104. The desired level of negative pressure can be pressure set or selected by the user. The pump controller 370 can control the pump (for example, pump motor) using pulse-width modulation (PWM) or pulsed control. A control signal for driving the pump can be a 0-100% duty cycle PWM signal. The pump controller 370 can perform flow rate calculations and detect alarms. The pump controller 370 can communicate information to the main controller 310. The pump controller 370 can be a low-power processor.

Any of the one or more communications controllers 330 can provide connectivity (such as, a wired or wireless connection 332). The one or more communications controllers 330 can utilize one or more transceivers 340 for sending and receiving data. The one or more transceivers 340 can include one or more antennas, optical sensors, optical transmitters, vibration motors or transducers, vibration sensors, acoustic sensors, ultrasound sensors, or the like. Any of the one or more transceivers 340 can function as a communications controller. In such case, the one or more communications controllers 330 can be omitted. Any of the one or more transceivers 340 can be connected to one or more antennas that facilitate wireless communication. The one or more communications controllers 330 can provide one or more of the following types of connections: Global Positioning System (GPS), cellular connectivity (for example, 2G, 3G, LTE, 4G, 5G, or the like), NFC, Bluetooth connectivity (or BLE), radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software or firmware, pairing, and the like.

Any of the one or more communications controllers 330 can provide dual GPS/cellular functionality. Cellular functionality can, for example, be 3G, 4G, or 5G functionality. The one or more communications controllers 330 can communicate information to the main controller 310. Any of the one or more communications controllers 330 can include internal memory or can utilize memory 350. Any of the one or more communications controllers 330 can be a low-power processor.

The control system 300 can store data, such as GPS data, therapy data, device data, and event data. This data can be stored, for example, in memory 350. This data can include patient data collected by one or more sensors. The control system 300 can track and log therapy and other operational data. Such data can be stored, for example, in the memory 350.

Using the connectivity provided by the one or more communications controllers 330, the control system 300 can upload any of the data stored, maintained, or tracked by the control system 300 to a remote computing device, such as the device 334. The control system 300 can also download various operational data, such as therapy selection and parameters, firmware and software patches and upgrades, and the like (for example, via the connection to the device 334). The one or more additional processors 380, such as processor for controlling one or more user interfaces (such as, one or more displays), can be utilized. In some cases, any of the illustrated or described components of the control system 300 can be omitted depending on an embodiment of a wound monitoring or treatment system in which the control system 300 is used.

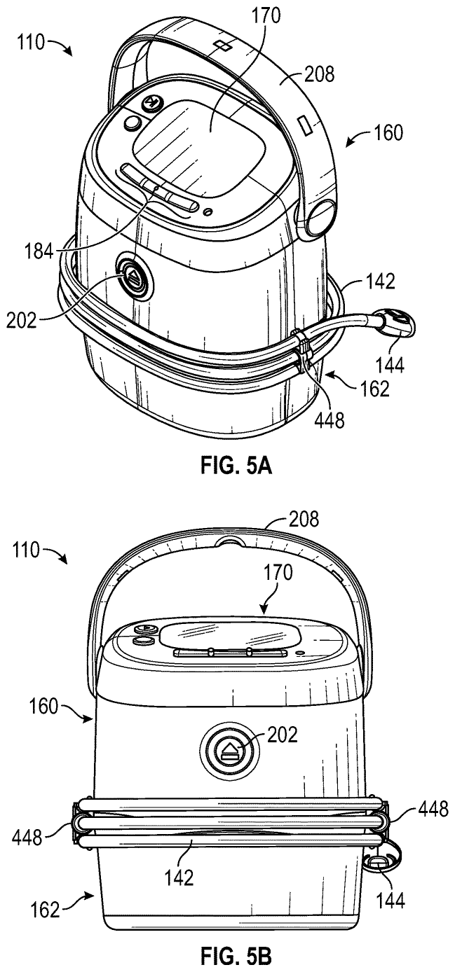

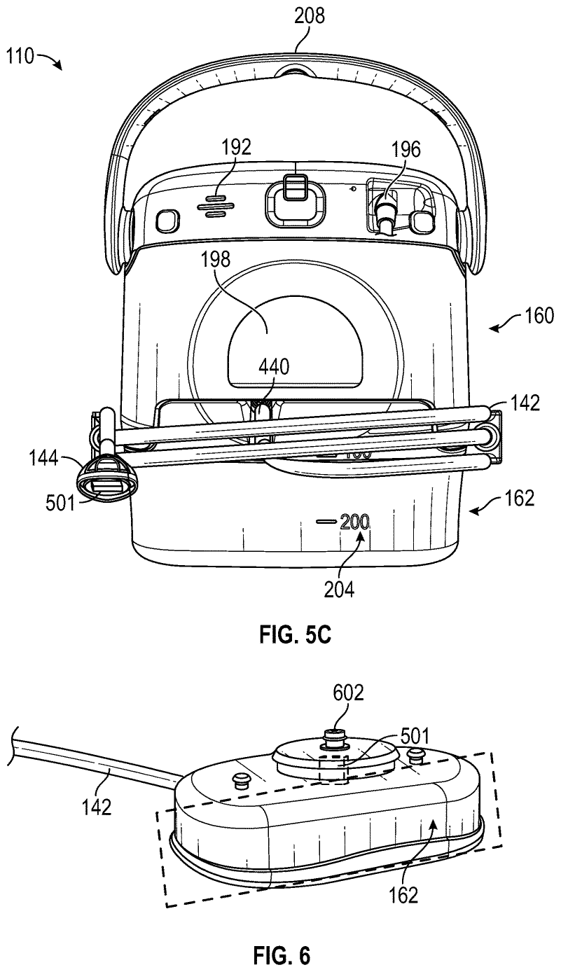

FIGS. 5A-5C show an arrangement of a negative pressure wound therapy device, which can be the device 110. The pump assembly 160 can have a modular design, in that many of the subcomponents of the pump assembly 160 are grouped and designed to be in modules. This modular arrangement of the various components of the pump assembly 160 can make it easier and quicker to remove and replace any failed components of the pump assembly 160.

As illustrated, the pump assembly 160 and canister 162 can be connected, thereby forming the wound therapy device 110. The pump assembly 160 can include a user interface, communications interfaces, negative pressure generation and control, and alarms generation. Some arrangements of the pump assembly 160 can include a housing, the main function of which is to enclose or house the electronics and other components. The housing can provide patient safety and isolation from the device internals, can protect the pump device against impact damage, and can provide aesthetic appeal. The housing can also provide a clear window to allow the user to view the display. The main housing can be easily removed without disturbing the rest of the device and component connections (e.g., can be removed without requiring any electrical connectors to be disconnected), making for simpler repairs.

The pump assembly 160 can also include a core assembly. Some arrangements of the core assembly can include all or mostly all of the electrical and mechanical components and features required for the user interface, negative pressure control, and battery operation. In some arrangements, the core assembly can be the central sub-assembly of the pump assembly and can be configured to be an easily extractable part from the pump assembly to make service and repair easier and faster.

The core assembly can be separated out into separate components easily for ease of service, cleaning, and assembly. The pump assembly 160 can also include a rear trim panel, which can provide a USB interface, a charger port, and a speaker. The rear trim panel can be easily removed (e.g., by removing two screws and associated screw cover plates) to allow for quick repairs to the charger connector, which can be broken through device misuse. The canister inlet tube 142 can be coupled with a dressing port connector 144 that can be used to connect with the conduit 108 of FIG. 1B. A turbine assembly 501 can be positioned in the dressing port connector 144.

In some arrangements, the canister inlet tubing 142 that is connected with the canister 162 can have a length that is sufficient to connect with a dressing or port that is at least two feet, or at least three feet, or between one foot and three feet or more away from the canister 162. The canister inlet tubing 142 can be coupled with a connector 440 that extends away from a surface of the canister 162. The connector 440 can be in fluid communication with an interior space within the canister body, and can be configured to couple with an end portion of the canister inlet tubing 142. All or a portion of the canister inlet tubing 142 can wrap around the canister 162 when all or a portion of the canister inlet tubing 142 is not needed.

Any of the negative pressure wound therapy devices described herein can include one or more features disclosed in U.S. Pat. No. 9,737,649, U.S. Patent Publication No. 2017/0216501, International Patent Publication No. WO 2022/200453, or International Patent Application No. PCT/EP2022/079091, each of which is incorporated by reference in its entirety.

### Multiple Dressing Negative Wound Therapy

FIG. 4 illustrates another negative pressure wound treatment system 400. The system 400 can include a wound therapy device capable of supplying negative pressure to the wound site or sites, such as wound therapy device 110. The wound therapy device 110 can be in fluidic communication with one or more wound dressings 406a, 406b (collectively referred to as 406) so as to supply negative pressure to one or more wounds, such as the wounds 104a and 104b. A first fluid flow path can include components providing fluidic connection from the wound therapy device 110 to the first wound dressing 406a. As a non-limiting example, the first fluid flow path can include the path from the wound dressing 406a to the wound therapy device 110 or the path from the first wound dressing 406a to an inlet 446 of a branching attachment (or connector) 444 in fluidic connection with the wound therapy device 110. Similarly, a second fluid flow path can include components providing fluidic connection from the wound therapy device 110 to the second wound dressing 406b.

The system 400 can be similar to the system 100 with the exception that multiple wounds 104a and 140b are being treated by the system 400. The system 400 can include any one or more of the components of the system 100, which are illustrated in FIG. 4 with appended letter “a” or “b” to distinguish between the first and second wounds (such as, the wounds 104a and 104b, the covers 106a and 106b). As illustrated, the system 400 can include a plurality of wound dressings 406a, 406b (and corresponding fluid flow paths) in fluidic communication with the wound therapy device 110 via a plurality of suction adapters, such as the adapter 108. The suction adapters can include any one or more of the components of the adapter 108, which are illustrated in FIG. 4 with appended letter “a” or “b” to distinguish between the first and second wounds (such as, the bridge portions 130a and 130b, the connectors 134a and 134b, and the caps 140a and 140b).

The wound therapy device 110 can be fluidically coupled via the canister inlet tube 142 with the inlet 446 of the connector 444. The connector 444 can be fluidically coupled via branches 445a, 445b and tubes or conduits 442a, 442b with the connectors 134a, 134b, which can be fluidically coupled with the tubes or conduits 130a, 130b. The tubes or conduits 130a, 130b can be fluidically coupled with the dressings 406a, 406b. Once all conduits and dressing components are coupled and operably positioned, the wound therapy device 110 can be activated, thereby supplying negative pressure via the fluid flow paths to the wounds 104a, 104b. As illustrated, the connector 444 can support the turbine assembly 501 (which can be positioned in the inlet 446) or the tubes or conduits 130a, 130b can support turbine assemblies 501 medially located within as well as the inlet 446. The location of the turbine assembly 501 is not limited to the illustration and the assembly can be located in one or more locations of the fluid flow paths. Application of negative pressure can be applied until a desired level of healing of the wounds 104a, 104b is achieved. Although two wounds and wound dressing are illustrated in FIG. 4, some implementations of the wound therapy device 110 can provide treatment to a single wound (for instance, by closing the unused branch 445a or 445b of the connector 444) or to more than two wounds (for instance, by adding branches to the connector 444).

The system 400 can include one or more features disclosed in U.S. Patent Publication No. 2020/0069850, or International Publication No. WO2018/167199, each of which is incorporated by reference in its entirety.

### Turbine Assembly

One or more parameters of negative pressure wound therapy can be monitored using a turbine assembly positioned in the fluid flow path. These parameters include flow rate in the fluid flow path, viscosity of fluid (or type of fluid), pressure drop, level of fluid (for instance, in the canister), blockage, or leak, among others. Advantageously, efficiency of negative pressure wound therapy can be improved and patient safety can be promoted.

FIG. 6 illustrates a cross-sectional view of the canister 162. The canister 162 can support the turbine assembly 501 that can be utilized to monitor the flow rate in the fluid flow path, monitor a viscosity (and type) of fluid in the fluid flow path, monitor the volume of fluid within the canister 162, among others. The turbine assembly 501 can be positioned at or near an inlet 602 of the canister 162. As described herein, in some cases, the turbine assembly 501 can be positioned within the canister 162, one or more connectors, one or more tubing or lumens, or one or more wound dressing ports.

The turbine assembly 501 can be inserted or attached to component of the negative pressure wound therapy system, integrated within a component, be placed within a cavity of a component (such as, the canister, tubing or lumen, or dressing port). The turbine assembly 501 can be inserted or attached via an interference fit, mechanical fixture (such as, a snap-fit mechanism), adhesive bond, magnetically or through creating a permanent fixture (such as, welding). The turbine assembly 501 can be integrated via over molding or by any of method stated in this paragraph or elsewhere in the description.

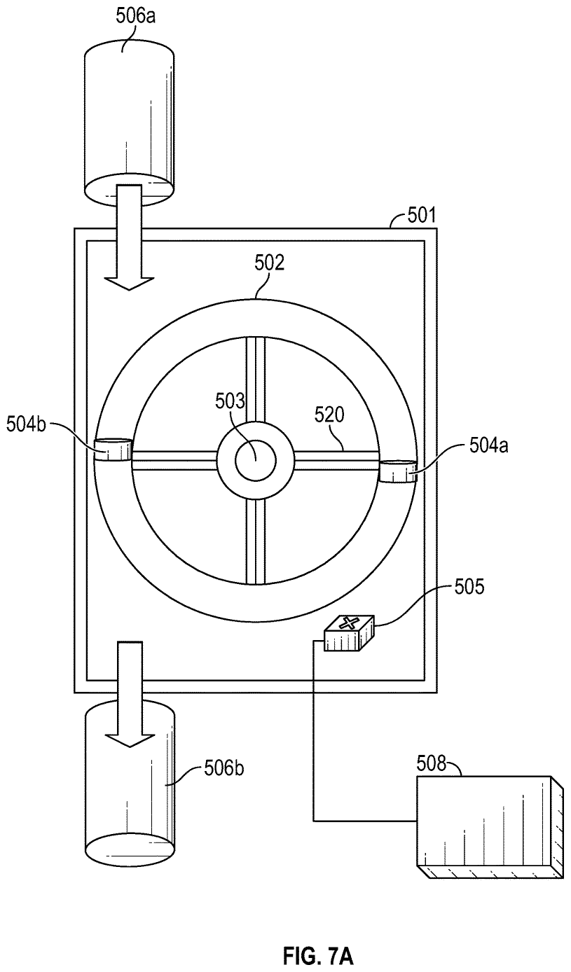

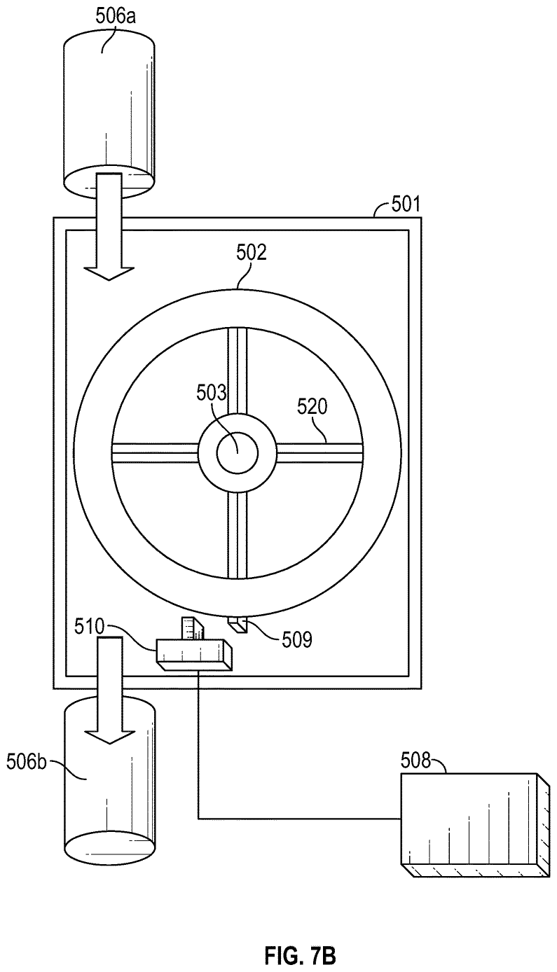

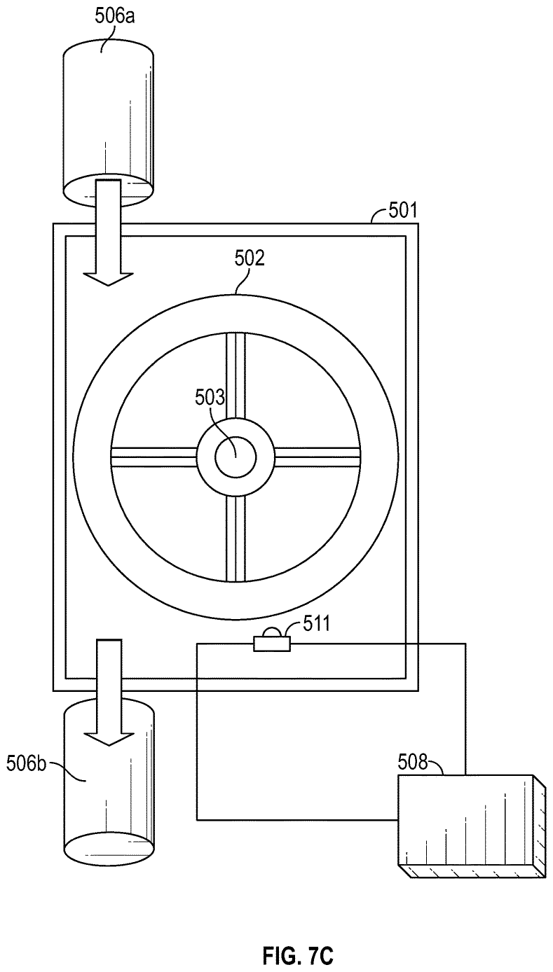

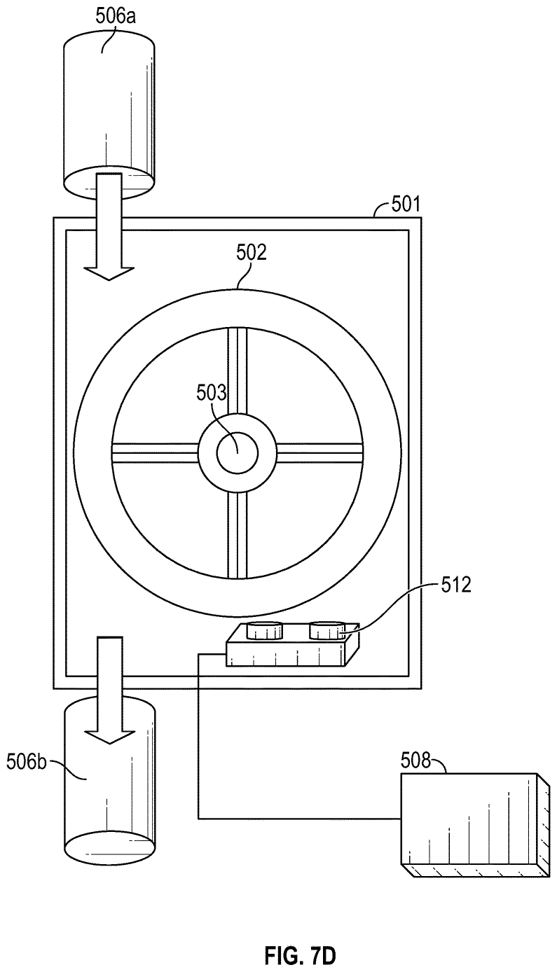

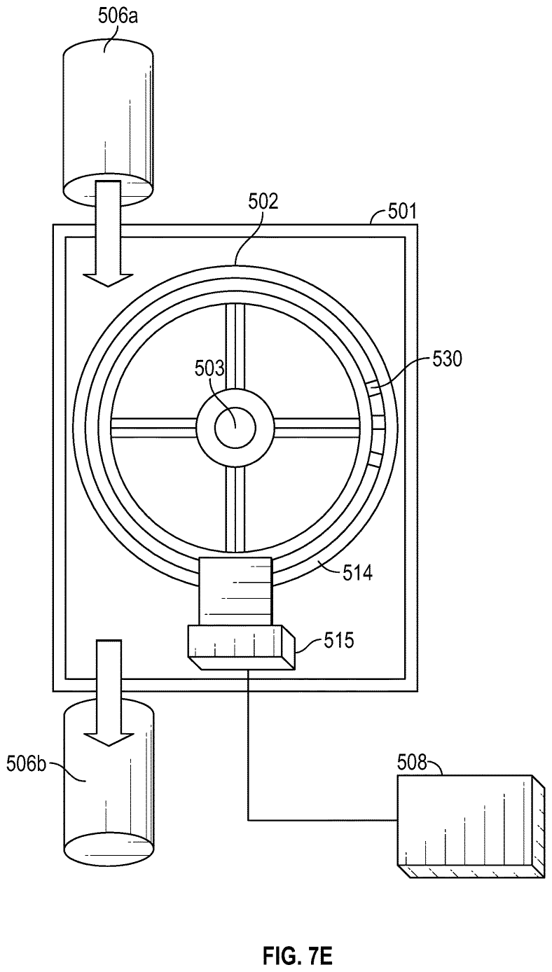

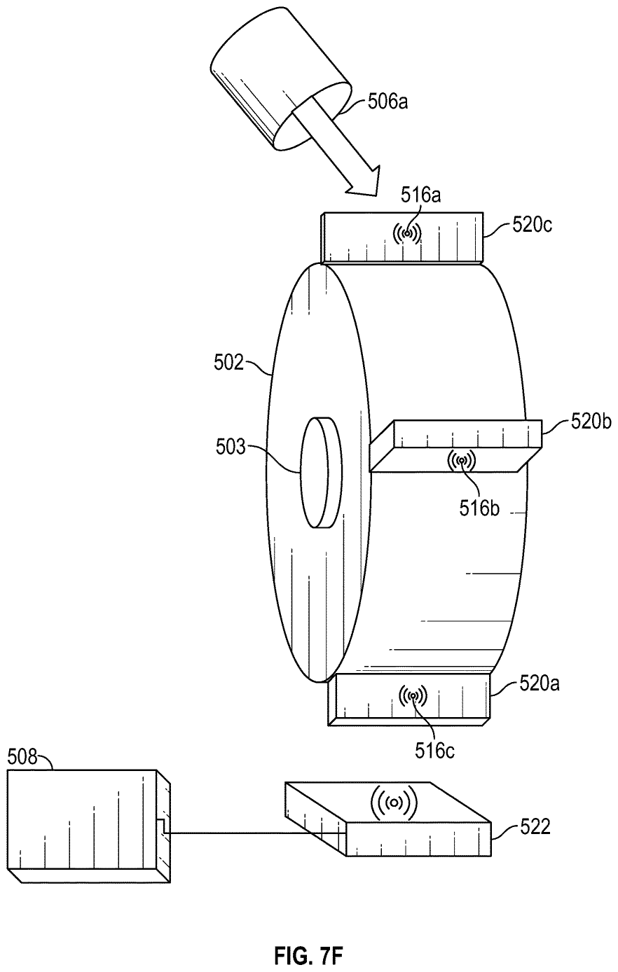

As described herein, the turbine assembly 501 can include (or cooperate with) one or more sensors (such as, magnetic sensors, conductive sensors, optical sensors, proximity sensors, etc.) that can sense the rotation of the turbine assembly. With reference to FIG. 7A, the turbine assembly 501 can include a turbine wheel 502 with one or more fins 520 (or spokes). In operation, fluid flowing through the fluid flow path (such as, fluid aspirated from the wound) passes over the fins 520 (such as, the fins 520a, 520b, 520c of FIG. 7F) of the turbine wheel 502 and rotates the wheel. The flow of the fluid can create a force at which the turbine wheel 502 can overcome static friction of a motionless turbine wheel 502. The speed of rotation of the turbine wheel 502 can be directly related to the flowrate of the fluid in the fluid flow path.

Conduits 506a and 506b illustrate a portion of the fluid flow path via which the fluid flows through the turbine assembly 501. The turbine wheel 502 can be made of a low surface energy material to minimize adhesive contact of the fluid passing over the wheel. The turbine wheel 502 can be supported by a bearing 503 (which can be made, for instance, from polyethylene). The turbine wheel 502 can be loosely supported by the bearing 503, which can permit making the turbine wheel 502 as large as possible to reduce losses due to friction.

In some implementations, the turbine assembly 501 can utilize magnetic sensors to monitor the speed of rotation and, correspondingly, the flow rate. With reference to FIG. 7A, magnets 504a and 504b can be attached to the fins 520 of the turbine wheel 502. Changes in the magnetic field caused by the rotation of the turbine wheel 502 can be detected by a Hall effect sensor 505. For instance, as one of the magnets 504a or 504b passes proximal to the Hall effect sensor 505, the sensor can detect presence of the magnetic field. The magnets 504a and 504b can be equally spaced on the turbine wheel 502 to provide optimal rotational balance of the turbine wheel 502 as well as maintain the center of mass. In some cases, more than two magnets can be used or a single magnet can be used. The Hall effect sensor 505 can detect that one of the magnets 504a, 504b has passed the sensor 505 and monitor the speed of rotation of the wheel 502. The sensor 505 can be connected with electronic processing circuitry 508, which can be part of the control system 300. The processing circuitry 508 can interpret data provided by the sensor 505 and determine the speed of rotation and the flow rate. In the illustrated implementation or any of the implementations disclosed herein, time between sensor activations can be indicative of the speed of rotation, which is directly proportional to the flow rate.

FIG. 7B illustrates a turbine assembly 501 with a mechanical triggering system. The turbine wheel 502 can be fitted with one or more protrusions 509, which can be spaced apart at equal distances. A switch 510 can be activated (such as, bent) when being contacted by the one or more protrusions 509. The one or more protrusions 509 can be positioned on the one or more fins 520 of the turbine wheel 502. This can facilitate triggering of the switch 510 at a specified frequency per rotation of the turbine wheel 502. As the turbine wheel 502 is rotated as a result of the fluid flow, the one or more protrusions 509 activate the switch 510 upon a full or partial rotation of the turbine wheel 502. The switch 510 can be positioned proximal to the turbine wheel 502 or vice versa. The switch 510 can provide data to the electronic circuitry 508 that can determine the speed of rotation and the flow rate.

FIG. 7C illustrates a turbine assembly 501 with photo sensing system. A photo sensor 511 (such as, a photoelectric sensor) can be located in close proximity to the turbine wheel 502. As the turbine wheel 502 is rotated due to the fluid flow, the photoelectric sensor 511 can detect one or more color patterns positioned on the turbine wheel 502 (for instance, on the one or more fins 520). The one or more color patterns can be images or text. In some implementations, one or more light sources (such as, LED, laser, etc.) can be positioned on the turbine wheel 502 and emitted light can be detected by the photo sensor 511. The emitted light can be colored, pulsed, or the like. One or more color patterns or emitted light can be sensed upon a full or partial revolution of the turbine wheel 502. The photo sensor 511 can provide data to the electronic circuitry 508 that can determine the speed of rotation and the flow rate. In some cases, the sensor 511 can be located on the turbine wheel 502 and the one or more color patterns or light sources can be positioned proximal to the wheel.

The photo sensor 511 can be an infrared sensor that detects infrared light emitted by one or more sources located on the turbine wheel 502. In some cases, the photo sensor 511 can be positioned on the turbine wheel 502, and the one or more infrared light sources can be positioned proximal to the turbine wheel 502. Generally, the photo sensor 511 can be a radiation sensor configured to detect radiation emitted by one or more radiation sources during rotation of the turbine wheel 502.

FIG. 7D illustrates a turbine assembly 501 with a proximity sensing system. A proximity sensor 512 can be a capacitive sensor, inductive sensor, optical sensor, ultrasonic sensor, magnetic sensor, among others. The proximity sensor 512 can sense rotation of the turbine wheel 502. The proximity sensor 512 can provide data to the electronic circuitry 508 that can determine the speed of rotation and the flow rate.

FIG. 7E illustrates a turbine assembly 501 with an electrically conductive sensing system. An electrically conductive strip 514 can be adhered to the turbine wheel 502. A sensor 515 (such as, an electrode) can be in contact with the conductive strip 514. One or more gaps 530 can be placed along the conductive strip 514. As the turbine wheel 502 is rotated due to the flow of fluid, the sensor 515 in contact with the conductive strip 514 can sense electrical field changes (or pulses) from the one or more gaps 530, which can be indicative of a full or partial rotation of the turbine wheel 502. The sensor 515 can provide data to the electronic circuitry 508 that can determine the speed of rotation and the flow rate. Similarly, a change in resistance, capacitance, inductance, or the like can be detected.

FIG. 7F illustrates a turbine assembly 501 with a communication tag sensing system. One or more communication tags can be positioned on the turbine wheel 502. For example, three tags 516a, 516b, 516c can be positioned on the turbine wheel 502, such as on the fins 520a, 520b, or 520c. The one or more communication tags can communicate using NFC, RFID, Bluetooth, BLE, or the like. For instance, low frequency (such as, 125-134 kHz), high frequency (such as, 13.56 MHz), or ultra high frequency (such as, 856-960 MHz) tags can be utilized. As the turbine wheel 502 is rotated due to the flow of fluid, the tags 516a, 516b, and 516c interact with a reader 522 that detects the tags (such as, detects one or more signals transmitted by the tags). As a result, a partial or full rotation of the turbine wheel 502 can be detected. The reader 522 can provide data to the electronic circuitry 508 that can determine the speed of rotation and the flow rate. As described herein, the tags 516a, 516b, 516c can be equally spaced apart to provide optimal rotational balance of the turbine wheel 502 as well as maintain a center of mass.

Rotation of the turbine wheel 502 of the turbine assembly 501 can generate electric power. The amount of power generated can vary based on the speed of rotation, which in turn can vary depending on the viscosity of fluid flowing in the fluid flow path. For instance, flow of fluid with higher viscosity (such as, blood) can flow slower than fluid with lower viscosity (such as, water), resulting in smaller amount of power being generated. Monitoring the generated power over time can be used to estimate the viscosity and type of fluid flowing in the fluid flow path. In some implementations, changes in the flow rate can be monitored to estimate the viscosity and type of fluid directly.

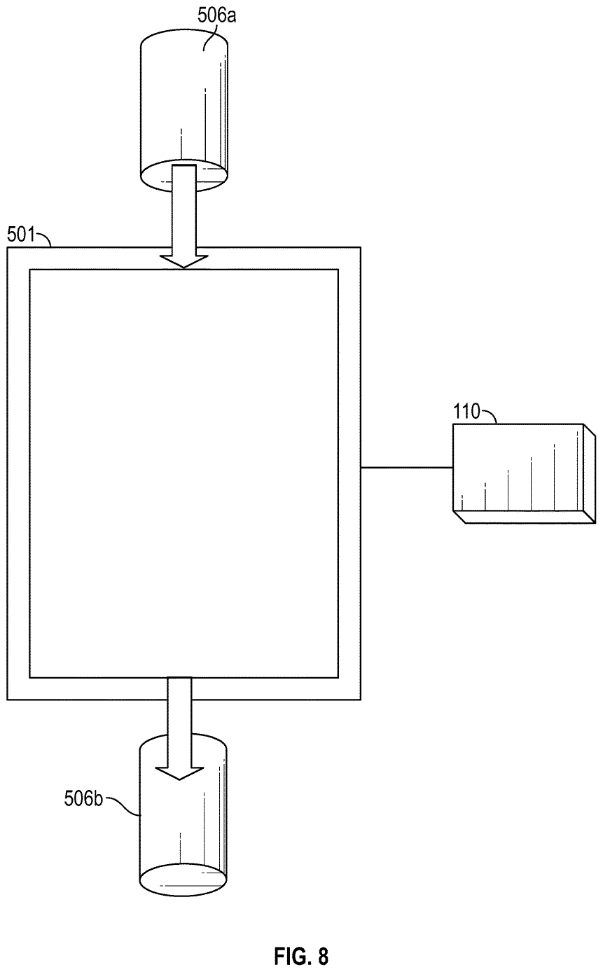

Generated power can be used to power one or more components of the negative pressure wound therapy device. With reference to FIG. 8, the turbine assembly 501 can be connected to the wound therapy device 110 and can provide power to one or more electrical components, such as one or more components of the control system 300. Power can be provided to one or more sensors, such as pressure, temperature, color, impedance, or pH sensors, among others. In some cases, instead of or in addition to providing direct power, the turbine assembly 501 can be utilized as indicator of when to read data collected by the one or more sensors. The turbine assembly 501 can provide power responsive to fluid flow and can serve as a fluid detector for collection of sensor data. As a result, accuracy and efficiency can be improved.

One or more sensors can be located within the canister, canister inlet, tubing, or dressing port of the negative pressure wound therapy system. Wound fluid can physically contact one or more sensors or be sampled via capillary action outside of the source of fluid flow. One or more sensors (or the processing circuitry 508) can be connected to the turbine assembly 501 via wires, ink tracks or traces (such as, silver ink tracks), or the like. The connection can be made within or on the surface of tubes, lumens, or wound ports, among others.

Fluid flow rate determined using the turbine assembly 501 can be utilized to determine a pressure drop in the fluid flow path, such as, across a known length of a fluid flow path tubing. As a result, pressure at the wound can be determined without using a dedicated pressure sensor at or proximal to the wound. Negative pressure wound therapy at a desired set point can be accurately and reliably delivered to the wound.

Without being limited to any particular theory, pressure drop can be determined using Poiseuille law (reproduced below) that relates a pressure drop (Δp) to the pipe length (L), pipe radius (R), viscosity (μ), and flow rate (Q). Pressure drop can be determined relative to know pressure measured by a pressure sensor, such as the pressure sensor 325. The pressure sensor can measure pressure at or near the inlet of the wound therapy device 110. In some implementations, pressure drop can be measured relative to a pressure set point being provided by the negative pressure source.

$$Δ p = \frac{8 μ LQ}{π {R}^{4}}$$

By identifying the quantity of liquid compared to gas in the fluid flow path, the pressure drop between the wound therapy device 110 and the wound can be quantified. This can be accomplished by characterizing the relationship between the pressure drop and liquid to air ratio at various pressure set points.

Flow rate monitored by the turbine assembly 501 can be used to determine a level of fluid in the canister 162 or determine that the canister 162 is full. The turbine assembly 501 can be supported by the canister 162 (such as, positioned in the canister inlet or in the interior volume of the canister). The turbine wheel 502 can rotate about an axis parallel or perpendicular to the fluid level within the canister 162. As the canister 162 is filled to the maximum capacity with wound exudates and other fluids, when the level of fluid in the canister reaches a maximum level, the turbine wheel 502 of the sensor assembly 501 can no longer spin. Lack of rotation of the turbine wheel 502 can indicate that the canister 162 is filled to capacity.

Additionally or alternatively, flow rate monitored by the turbine assembly 501 can be used to determine the level (or volume) of fluid in the canister 162. Monitoring the flow rate over a time duration can be used to determine a fluid volume collected in the canister 162 over the time duration. Accordingly, fluid level in the canister 162 can be accurately determined. Using a known canister volume, canister full or pre-full condition can be reliably determined and an indication can be. Estimate of when the canister will likely fill up and would need to be replaced can be made accurately.

An indication of any of the conditions monitored using the turbine assembly 501 can be provided using any of the approaches described herein. Patient safety can be improved, for instance, by detecting presence of blood in the fluid flow path (based on determining the viscosity and type of fluid as described herein) and stopping negative pressure wound therapy in response to detecting blood.

In some implementations, multiple turbine assemblies 501 can be positioned in different locations in the fluid flow path. This can permit determining a blockage downstream of the canister, distinguish such downstream blockage from canister full, or identify a leak in the fluid flow path (that is, flow rate that satisfies a particular threshold), among others.

Advantageously, the described approaches provide the ability to derive information relating to fluid passing in the fluid flow path. Characteristics of fluid aspirated from the wound can be measured prior to the fluid collecting in the canister, where its properties may change after contacting the gelling agent. Monitoring the flow rate of fluid aspirated from the wound can indicate whether the wound is healing, as would be indicated by a reduction in flow rate as the wound is healing. Other variables such as temperature, color, impedance, pH, or viscosity could also be used to infer a progression in the condition of the wound.

### Other Variations

Although some embodiments describe negative pressure wound therapy, the systems, devices, and/or methods disclosed herein can be applied to other types of therapies usable standalone or in addition to TNP therapy. Systems, devices, and/or methods disclosed herein can be extended to any medical device, and in particular any wound monitoring and/or treatment device. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. As another example, systems, devices, and/or methods disclosed herein can be used with a wound debridement system, patient monitoring system, or the like. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A negative pressure wound therapy system, comprising:
a source of negative pressure configured to be fluidically connected by a fluid flow path to a wound covered by a wound dressing and further configured to provide negative pressure to the wound;
a canister configured to be positioned in the fluid flow path and further configured to store fluid aspirated from the wound as a result of negative pressure being provided to the wound by the source of negative pressure; and
a turbine positioned within either the canister, a lumen, or a wound dressing port, at least one of the lumen or the wound dressing port configured to connect the source of negative pressure to the wound, the turbine further configured to facilitate monitoring a rate of a flow of fluid in the fluid flow path responsive to a rotation of the turbine caused by the flow of fluid.

2. The negative pressure wound therapy system of claim 1, wherein the turbine comprises a wheel that includes a plurality of fins.

3. The negative pressure wound therapy system of claim 2, further comprising:
at least two magnets attached to the plurality of fins; and
a Hall effect sensor configured to interact with the at least two magnets to monitor a rotational speed of the turbine,
wherein the rate of flow of fluid is monitored responsive to detection of the rotational speed.

4. The negative pressure wound therapy system of claim 3, wherein the at least two magnets are spaced equally on the wheel of the turbine.

5. The negative pressure wound therapy system of claim 1, wherein the rotation of the turbine causes an energy to be produced, an amount of the energy being dependent on a viscosity of fluid flowing in the fluid flow path, and wherein the system further comprises an electronic processing circuitry configured to determine the viscosity of fluid based on the amount of the energy.

6. (canceled)

7. The negative pressure wound therapy system of claim 1, further comprising electronic processing circuitry configured to provide an indication of the rate of flow of fluid.

8. The negative pressure wound therapy system of claim 1, further comprising an electronic processing circuitry configured to determine a pressure at the wound based on the rate of flow of fluid and provide an indication of the pressure at the wound.

9. The negative pressure wound therapy system of claim 8, wherein the electronic processing circuitry is configured to determine the pressure at the wound based on the rate of flow of fluid, a viscosity of fluid flowing in the fluid flow path, and a level of negative pressure being at least one of provided by the source of negative pressure to the wound or measured at a location in the fluid flow path.

10. The negative pressure wound therapy system of claim 9, wherein the rotation of the turbine causes an energy to be produced, an amount of the energy being dependent on the viscosity of fluid flowing in the fluid flow path, and wherein the electronic processing circuitry is further configured to determine the viscosity of fluid based on the amount of the energy.

11. The negative pressure wound therapy system of claim 9, wherein the electronic processing circuitry is configured to determine the pressure at the wound further based on a length, radius, and cross-sectional area of the lumen.

12. The negative pressure wound therapy system of claim 1, wherein the turbine is positioned in an inlet of the canister, and wherein the system further comprises an electronic processing circuitry configured to:
determine that the canister is full responsive to detecting a lack of the rotation of the turbine, wherein an axis of the rotation of the turbine is either parallel or perpendicular to a level of fluid in the canister; and
provide an indication that the canister is full.

13. (canceled)

14. The negative pressure wound therapy system of claim 1, further comprising an electronic processing circuitry configured to:
determine a level of fluid in the canister based on the rate of flow of fluid; and
provide an indication of the level of fluid in the canister.

15. (canceled)

16. The negative pressure wound therapy system of claim 14, wherein the electronic processing circuitry is further configured to determine that the canister is full responsive to determining that the level of fluid in the canister has reached a threshold fluid level.

17. The negative pressure wound therapy system of claim 14, wherein the electronic processing circuitry is configured to determine the level of fluid in the canister based on monitoring the rate of flow of fluid over a duration of time.

18. A negative pressure wound therapy system, comprising:
a source of negative pressure configured to be fluidically connected by a fluid flow path to a wound covered by a wound dressing and further configured to provide negative pressure to the wound; and
a turbine positioned within either a canister, a lumen, or a wound dressing port, the canister configured to store fluid aspirated from the wound, at least one of the lumen or the wound dressing port configured to connect the source of negative pressure to the wound, the turbine further configured to facilitate monitoring a rate of a flow of fluid in the fluid flow path responsive to a rotation of the turbine caused by the flow of fluid.

19. The negative pressure wound therapy system of claim 18, wherein the rotation is monitored in response to at least one of:
a change in a magnetic field caused by movement of a magnet attached to the turbine;
a change in an electric field caused by movement of a conductive component positioned on the turbine;
a change in a resistance caused by movement of the turbine;
a movement of a wireless tag attached to the turbine;
a movement of a protrusion of the turbine;
a change of color; or
a movement of an infrared emitter or sensor supported by on the turbine.

20. The negative pressure wound therapy system of claim 18, wherein:
the turbine comprises a wheel that includes a plurality of fins;
at least two magnets are attached to the plurality of fins; and
the system comprises a Hall effect sensor configured to interact with the at least two magnets to monitor a rotational speed of the turbine,
wherein the rate of flow of fluid is monitored responsive to detection of the rotational speed.

21. The negative pressure wound therapy system of claim 18, wherein the rotation of the turbine causes an energy to be produced, an amount of the energy being dependent on a viscosity of fluid flowing in the fluid flow path, and wherein the system further comprises an electronic processing circuitry configured to determine the viscosity of fluid based on the amount of energy.

22. The negative pressure wound therapy system of claim 18, further comprising an electronic processing circuitry configured to:
determine a pressure at the wound based on the rate of flow of fluid; and
provide an indication of the pressure at the wound.

23. The negative pressure wound therapy system of claim 18, further comprising:
the canister; and
an electronic processing circuitry configured to at least one of:
determine that the canister is full responsive to detecting a lack of the rotation of the turbine; or
determine a level of fluid in the canister based on the rate of flow of fluid.

24. (canceled)
